(19) United States
(12) Patent Application Publication
(10) Pub. No.: US 2007/0238711 A1
(43) Pub. Date: Oct. 11, 2007

(54) Combination Therapy with Glatiramer Acetate and Minocycline for the Treatment of Multiple Sclerosis

(76) Inventors: Luanne Metz, Calgary (CA); Jean Godin, Montreal (CA); Voon Wee Yong, Calgary (CA)

Correspondence Address: COOPER & DUNHAM, LLP, 1185 AVENUE OF THE AMERICAS, NEW YORK, NY, 10036, US

(21) Appl. No.: 11/597,571

(22) PCT Filed: May 27, 2005
(86) PCT No.: PCT/US05/18938

§ 371 Date: May 2, 2007

Related U.S. Application Data
(60) Provisional application No. 60/575,114, filed on May 28, 2004.

(51) Int. Cl. A61K 31/60; A61P 21/00

(52) U.S. Cl. 514/159

(57) ABSTRACT

The subject invention provides a method of treating a subject afflicted with a form of multiple sclerosis comprising periodically administering to the subject an amount of glatiramer acetate and an amount of minocycline, wherein the amounts when taken together are effective to alleviate a symptom of the form of multiple sclerosis in the subject so as to thereby treat the subject. Additionally, the subject invention provides a pharmaceutical composition comprising an amount of glatiramer acetate and an amount of minocycline, wherein the amounts when taken together are effective to alleviate a symptom of a form of multiple sclerosis in a subject. The subject invention also provides a package comprising glatiramer acetate, minocycline and instructions for use of the together to alleviate a symptom of a form of multiple sclerosis in a subject. The subject invention further provides a pharmaceutical combination comprising separate dosage forms of an amount of glatiramer acetate and an amount of minocycline, which combination is useful to alleviate a symptom of a form of multiple sclerosis in a subject.

This application claims the benefit of U.S. Provisional Application No. 60/575,114, filed May 28, 2004, the contents of which are hereby incorporated by reference.

Throughout this application, various events are referenced in parenthesis. The disclosures of these publications in their entireties are hereby incorporated by reference into this application in order to more fully describe the state of the art to which this invention pertains.

### FIELD OF THE INVENTION

The subject invention relates to combination therapy for treating multiple sclerosis.

### BACKGROUND OF THE INVENTION

One of the more common neurologic disorders in human adults is multiple sclerosis. This condition is a chronic, inflammatory CNS disease characterized pathologically by demyelination. There are five main forms of multiple sclerosis: 1) benign multiple sclerosis; 2) relapsing-remitting multiple sclerosis (RR-MS); 3) secondary progressive multiple sclerosis (SP-MS); 4) primary progressive multiple sclerosis (PP-MS); and 5) progressive-relapsing multiple sclerosis (PR-MS). Benign multiple sclerosis is characterized by 1-2 exacerbations with complete recovery, no lasting disability and no disease progression for 10-15 years after the initial onset. Benign multiple sclerosis may, however, progress into other forms of multiple sclerosis. Patients suffering from RR-MS experience sporadic exacerbations or relapses, as well as periods of remission. Lesions and evidence of axonal loss may or may not be visible on MRI for patients with RR-MS. SP-MS may evolve from RR-MS. Patients afflicted with SP-MS have relapses, a diminishing degree of recovery during remissions, less frequent remissions and more pronounced neurological deficits than RR-MS patients. Enlarged ventricles, which are markers for atrophy of the corpus callosum, midline center and spinal cord, are visible on MRI of patients with SP-MS. PP-MS is characterized by a steady progression of increasing neurological deficits without distinct attacks or remissions. Cerebral lesions, diffuse spinal cord damage and evidence of axonal loss are evident on the MRI of patients with PP-MS. PR-MS has periods of acute exacerbations while proceeding along a course of increasing neurological deficits without remissions. Lesions are evident on MRI of patients suffering from PR-MS (Multiple sclerosis: its diagnosis, symptoms, types and stages, 2003<http://www.albany.net/˜tjc/multiple-sclerosis.html>).

Researchers have hypothesized that multiple sclerosis is an autoimmune disease (Compston, Genetic susceptibility to multiple sclerosis, in McAlpine's Multiple Sclerosis, Matthews, B. ed., London: Churchill Livingstone, 301-319, 1991; Hafler and Weiner, M S: A CNS and systemic autoimmune disease, Immunol. Today, 10:104-107, 1989; Olsson, Immunology of multiple sclerosis, Curr. Opin. Neurol. Neurosurg., 5:195-202, 1992). An autoimmune hypothesis is supported by the experimental allergic encephalomyelitis (EAE) model of multiple sclerosis, where the injection of certain myelin components into genetically susceptible animals leads to T cell-mediated CNS demyelination (Parkman, Graft-versus-host disease, Ann. Rev. Med., 42:189-197, 1991). Another theory regarding the pathogenesis of multiple sclerosis is that a virus, bacteria or other agent, precipitates an inflammatory response in the CNS, which leads to either direct or indirect (“bystander”) myelin destruction, potentially with an induced autoimmune component (Lampert, Autoimmune and virus-induced demyelinating diseases. A review, Am. J. Path., 91:176-208, 1978; Martyn, The epidemiology of multiple sclerosis in McAlpine's Multiple Sclerosis, Matthews, B., ed., London: Churchil Livingstone, 3-40, 1991). Another experimental model of multiple sclerosis, Theiler's murine encephalomyelitis virus (TMEV)(Dal Canto and Lipton, Multiple sclerosis. Animal model: Theiler's virus infection in mice, Am. J. Path. 88:497-500, 1977; Rodriguez, et al., Theiler's murine encephalomyelitis: a model of demyelination and persistence of virus, Crit. Rev. Immunol., 7:325, 1987), supports the theory that a foreign agent initiates multiple sclerosis. In the TMEV model, injection of the virus results in spinal cord demyelination.

Glatiramer acetate (GA), also known as Copolymer-1, has been shown to be effective in treating multiple sclerosis (MS) (Lampert, Autoimmune and virus-induced demyelinating diseases. A review, Am. J. Path., 91:176-208, 1978; Martyn, The epidemiology of multiple sclerosis in McAlpine's Multiple Sclerosis, Matthews, B., ed., London: Churchil Livingstone, 3-40, 1991). Daily subcutaneous injections of glatiramer acetate (20 mg/injection) reduce relapse rates, progression of disability, appearance of new lesions by magnetic resonance imaging (MRI), (Johnson, et al., Copolymer 1 reduces relapse rate and improves disability in relapsing-remitting multiple sclerosis: results of a phase III multicenter, double-blind placebo-controlled trial, The Copolymer 1 Multiple Sclerosis Study Group, Neurol., 45:1268, 1989) and appearance of “black holes” (Filippi, et al., Glatiramer acetate reduces the proportion of MS lesions evolving into black holes, Neurol., 57:731-733, 2001).

COPAXONE® is the brand name for a formulation containing glatiramer acetate as the active ingredient. Glatiramer acetate is approved for reducing the frequency of relapses in relapsing-remitting multiple sclerosis. Glatiramer acetate consists of the acetate salts of synthetic polypeptides containing four naturally occurring amino acids: L-glutamic acid, L-alanine, L-tyrosine, and L-lysine with an average molar fraction in COPAXONE® of 0.141, 0.427, 0.095 and 0.338, respectively. In COPAXONE®, the average molecular weight of the glatiramer acetate is 4,700-11,000 daltons. Chemically, glatiramer acetate is designated L-glutamic acid polymer with L-alanine, L-lysine and L-tyrosine, acetate (salt). Its structural formula is:
(Glu,Ala,Lys,Tyr)ₓ.CH₃COOH(C₅H₉NO₄.C₃H₇NO₂.C₆H₁₄N₂O₂.C₉H₁₁NO₃)ₓ.χC₂H₄O₂ CAS—147245-92-9.

The recommended dosing schedule of COPAXONE® for relapsing-remitting multiple sclerosis is 20 mg per day injected subcutaneously (“COPAXONE®” in Physician's Desk Reference, Medical Economics Co., Inc., Montvale, N.J., 2003, 3214-3218; see also U.S. Pat. Nos. 3,849,550; 5,800,808; 5,858,964, 5,981,589; 6,048,898; 6,054,430; 6,214,791; 6,342,476; and 6,362,161, the content of all of which are hereby incorporated by reference).

The precise mechanisms by which glatiramer acetate prevents the development of experimental allergic encephalomyelitis (EAE) and ameliorates MS are not fully elucidated, but some important immunological aspects of these features have been studied. GA shows some cross reactivity with Myelin Basic Protein (MBP), mediated by both T-cells and antibodies. It binds to various Major Histocompatibility Complex (MHC) class II molecules on antigen-presenting cells (APC) and prevents them from binding to T-cells with several antigen-recognition properties (Fridkis-Hareli, M., et al., Direct binding of myelin basic protein and synthetic copolymer-1 to class II major histocompatibility complex molecules on living antigen presenting cells—specificity and promiscuity. Proc. Natl. Acad. Sci. (USA), 1994, 91: 4872-4876; Fridkis-Hareli, M., et al., Synthetic copolymer-1 and myelin basic protein do not require processing prior to binding to class II major histocompatility complex molecules on living antigen-presenting cells. Cell Immunol., 1995, 163: 229-236). In rodents, GA suppresses the encephalitogenic effects of auto reactive T-cells. Passive transfer of GA-reactive T-cells prevents the development of EAE induced in rats or mice by MBP, protolipid protein (PLP) or Myelin Oligodendrocyte Glycoprotein (MOG) (Aharoni, D., et al., T-suppressor hybridomas and interleukin-2-dependent lines induced by copolymer-1 or by spinal cord homogenate down-regulate experimental allergic encephalomyelitis. Eur. J. Immunol., 1993, 23: 17-25; Lahat, N., et al., Copolymer-1 (GA®)-deriven anti-inflammatory cascade in multiple sclerosis patients. J. Neurology, 1997, 244: Supp 3, S32; Comi G., et al., The Effect of Glatiramer Acetate (GA®) on MRI Detected Disease Activity in Subjects with R_R MS: A Multicenter, Randomized, Double-blind, Placebo-controlled Study Extended by Open-Label Treatment. Abstract 9003—ACTRIMIS—Basel). In humans, daily injection of GA, resulted in the development of a T helper 2 (Th2)-type of protective response over time. These activated GA-reactive T-cells, when reaching the site of injury, secrete cytokines associated with both Th2 (IL-4) profiles and neurotrophic factors such as Brain Derived Neurotrophic Factor (Ziemessen) (BDNF) serves a dual role: first exerting bystander suppression anti-inflammatory activity and later a neuroprotective action on axons.

Thus, GA has a dual mechanism of action. It is a unique immunomodulating agent that stimulates Th2 cells to secrete both anti-inflammatory cytokines as well as BDNF. This provides an anti-inflammatory milieu and neurotrophic support to the demyelinating axons protecting them from further degeneration over the long term. These features of GA are reflected in both the long-term efficacy of GA in reducing relapse rate as well as in affecting Magnetic Resonance Imaging (MRI) markers of axonal loss. This was demonstrated in a 9-month multicenter European and Canadian trial in patients with R-R MS comparing the effects of GA and placebo on magnetic resonance imaging (MRI) measures (Comi G., et al., A multinational, multi-center, randomized, double-blind, placebo-controlled study extended by open-label treatment to study the effect of glatiramer acetate (Copaxone) on disease activity as measured by cerebral magnetic resonance imaging in subjects with relapsing-remitting multiple sclerosis. Annals of Neurology; 44(3):507, 1998; Comi G., et al., Copaxone® MRI Study Group. The effect of glatiramer acetate (Copaxone®) on disease activity as measured by cerebral MRI in subjects with relapsing-remitting multiple sclerosis (RRMS): a multi-center, randomized, double-blind, placebo-controlled study extended by open-label treatment. Neurology; 52(6) Suppl. 2:A263-265, A289-A291, A336, A464, A491-A494, A496-A499, 1999). In this study, significantly fewer gadolinium-enhancing lesions progressed to persistent black holes in the GA-treated group than in the group receiving placebo. This suggests that GA may have the capacity to offer an axonal protective effect (Filippi et al., Glatiramer acetate reduces the proportion of MS lesions evolving into black holes, Neurol., 57:731-733, 2001).

Minocycline is a semisynthetic derivative of tetracycline, named [4S-(4∝,4a∝,5a∝,12a∝)]-4,7-bis(dimethylamino)-1,4,4a,5,5a,6,11,12a-octahydro-3,10,12,12a-tetrahydroxy-1,11-dioxo-2-naphthacenecarboxamide. Its structural formula is:

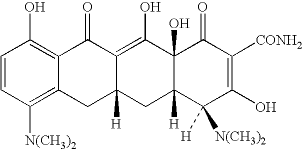

Minocycline is a tetracycline with antibacterial activity comparable to other tetracyclines with activity against a wide range of gram-negative and gram-positive organisms. The usual dosage and frequency of administration of minocycline differs from that of the other tetracyclines. The usual adult dosage of minocycline is 200 mg initially followed by 100 mg every 12 hours. For children above eight years of age, the usual dosage of minocycline is 4 mg/kg initially followed by 2 mg/kg every 12 hours (“MINOCIN®” in Physician's Desk Reference, Medical Economics Co., Inc., Montvale, N.J., 2003, 3420-3424).

Minocycline is commercially available as minoycline hydrochloride, C₂₃H₂₇N₃O₇, which has a molecular weight of 493.94, in a capsule under the tradename, DYNACIN®. Minocycline hydrochloride is also available under the tradename, MINOCIN®, as an oral suspension, a pellet-filled capsule or an intravenous preparation. However, the labeling for MINOCIN® intravenous specifically warns that parenteral therapy is indicated only when oral therapy is not adequate or tolerated. Oral therapy should be instituted as soon as possible. If intravenous therapy is given over prolonged periods of time, thrombophlebitis may result (“DYNACIN®” in Physician's Desk Reference, Medical Economics Co., Inc., Montvale, N.J., 2003, 1921-1923).

U.S. Patent Application Publication No. 2002/0022608, published Feb. 21, 2002 (Duncan et al.) disclosed a test of minocycline in an EAE model and suggested the use of minocycline to treat multiple sclerosis.

Minocycline has a wide range of immunomodulatory properties. When tested against EAE, minocycline has been, confirmed to dramatically suppress disease activity in chronic relapsing remitting EAE (Popovic et al., Inhibition of autoimmune encephalomyelitis by a tetracycline. Ann Neurol, 51: 215-223, 2002), to delay the course of severe EAE, and attenuate the severity of mild EAE (Brundula et al., Targeting leukocytes MMPs and transmigration: minocycline as a potential therapy for multiple sclerosis, Brain 125: 1297-1308, 20012). Specifically, minocycline suppresses clinical disease and histopatological evidence of inflammation within the CNS and prevents microglial activation and demyelination. The efficacy of minocycline in EAE is not thought to be due to its anti-microbial activity but rather due to some of its other reported actions, including the inhibition of production and activity of matrix metalloproteinases (Brundula), lowering of levels of various cytokines (Yrjanheikki, et al., A tetracycline derivative, minocycline, reduces inflammation and protects against focal cerebral ischemia with a wide therapeutic window. Proc Natl Acad Sci USA, 96: 13496-13500, 1999; Tikka T., et al., Minocycline provides neuroprotection against N-methyl-D-aspartate neurotoxicity by inhibiting microglia. J Immunol, 166 : 7527-7533, 2001; Wu, et al., Blockade of microglial activation is neuroprotective in the 1-methyl-4-phenyl-1,2,3,6-tetrahydropyridine mouse model of parkinson disease. J Neurosci, 22 (5): 1763-1771, 2002) and the attenuation of neural apoptosis (Arvin, et al., minocycline markedly protects the neonatal brain against hypoxic-ischemic injury. Ann Neurol, 52: 54-61, 2002; Zhu, et al., Minocycline inhibits cytochrome c release and delays progression of amyotrophic lateral sclerosis in mice. Nature, 417: 74-78, 2002).

However, minocycline has not been tested in combination with glatiramer acetate.

The administration of two drugs to treat a given condition, such as a form of multiple sclerosis, raises a number of potential problems. In vivo interactions between two drugs are complex. The effects of any single drug are related to its absorption, distribution, and elimination. When two drugs are introduced into the body, each drug can affect the absorption, distribution, and elimination of the other and hence, alter the effects of the other. For instance, one drug may inhibit, activate or induce the production of enzymes involved in a metabolic route of elimination of the other drug (Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics. Eval. and Res., Clin./Pharm., November 1999<http://www.fda.gov/cber/gdlns/metabol.pdf>). Thus, when two drugs are administered to treat the same condition, it is unpredictable whether each will complement, have no effect on, or interfere with, the therapeutic activity of the other in a human subject.

Not only may the interaction between two drugs affect the intended therapeutic activity of each drug, but the interaction may increase the levels of toxic metabolites (Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., November 1999<http://www.fda.gov/cber/gdlns/metabol.pdf>). The interaction may also heighten or lessen the side effects of each drug. Hence, upon administration of two drugs to treat a disease, it is unpredictable what change will occur in the negative side profile of each drug.

Additionally, it is accurately difficult to predict when the effects of the interaction between the two drugs will become manifest. For example, metabolic interactions between drugs may become apparent upon the initial administration of the second drug, after the two have reached a steady-state concentration or upon discontinuation of one of the drugs (Guidance for Industry. In vivo drug metabolism/drug interaction studies—study design, data analysis, and recommendations for dosing and labeling, U.S. Dept. Health and Human Svcs., FDA, Ctr. for Drug Eval. and Res., Ctr. for Biologics Eval. and Res., Clin./Pharm., November 1999<http://www.fda.gov/cber/gdlns/metabol.pdf>).

Thus, the success of one drug or each drug alone in an in vitro model, an animal model, or in humans, may not correlate into efficacy when both drugs are administered to humans.

In accordance with the subject invention, glatiramer acetate and minocycline are effective in combination to treat a form of multiple sclerosis, specifically, relapsing-remitting multiple sclerosis.

### SUMMARY OF THE INVENTION

The subject invention provides a method of treating a subject afflicted with a form of multiple sclerosis comprising periodically administering to the subject an amount of glatiramer acetate and an amount of minocycline, wherein the amounts when taken together are effective to alleviate a symptom of the form of multiple sclerosis in the subject so as to thereby treat the subject.

The subject invention further provides a pharmaceutical composition comprising an amount of glatiramer acetate and an amount of minocycline, wherein the amounts when taken together are effective to alleviate a symptom of a form of multiple sclerosis in a subject.

In addition, the subject invention provides a package comprising
i) a first pharmaceutical composition comprising an amount of glatiramer acetate and a pharmaceutically acceptable carrier;
ii) a second pharmaceutical composition comprising an amount of minocycline and a pharmaceutically acceptable carrier; and
iii) instructions for use of the first and second pharmaceutical compositions together to alleviate a symptom of a form of multiple sclerosis in a subject.

### DESCRIPTION OF THE DRAWINGS

FIG. 1 shows a timeline for the administration of glatiramer acetate, minocycline, MOG, pertussis toxin in Experiments 1-2.

FIG. 2 shows the average group clinical scores for manifestations of EAE in Experiment 1.

FIG. 3 shows the sum of clinical scores for EAE manifestations in Experiment 1.

FIG. 4 shows the average group clinical scores for EAE manifestations in Experiment 2.

FIG. 5 shows the sum of clinical scores for manifestations of EAE in Experiment 2.

### DETAILED DESCRIPTION OF THE INVENTION

The subject invention provides a method of treating a subject afflicted with a form of multiple sclerosis comprising periodically administering to the subject an amount of glatiramer acetate and an amount of minocycline, wherein the amounts when taken together are effective to alleviate a symptom of the form of multiple sclerosis in the subject so as to thereby treat the subject.

In one embodiment, the form of multiple sclerosis is relapsing-remitting multiple sclerosis.

In another embodiment, the subject is a human being.

In a further embodiment, each of the amount of glatiramer acetate when taken alone, and the amount of minocycline when taken alone is effective to alleviate the symptom of the form of multiple sclerosis.

In an embodiment, either the amount of glatiramer acetate when taken alone, the amount of minocycline when taken alone or each such amount when taken alone is not effective to alleviate the symptom of the form of multiple sclerosis.

In yet another embodiment, the symptom is the frequency of relapses, the frequency of clinical exacerbation, or the accumulation of physical disability.

In one embodiment, the amount of glatiramer acetate may be 10 to 80 mg; or 12 to 70 mg; or 14 to 60 mg; or 16 to 50 mg; or 18 to 40 mg; or 20 to 30 mg; or 20 mg. For each amount of glatiramer acetate, the amount of minocycline may be 50-200 mg; or 60-175 mg; or 70-150 mg; or 80-125 mg; or 90-110 mg; or 95-105 mg; or 100 mg.

Alternatively, the amount of glatiramer acetate may be in the range from 10 to 600 mg/week; or 100 to 550 mg/week; or 150 to 500 mg/week; or 200 to 450 mg/week; or 250 to 400 mg/week; or 300 to 350 mg/week; or 300 mg/week.

In another embodiment, the amount of glatiramer acetate may be in the range from 50 to 150 mg/day; or 60 to 140 mg/day; or 70 to 130 mg/day; or 80 to 120 mg/day; or 90 to 110 mg/day; or 100 mg/day.

Alternatively, the amount of glatiramer acetate may be in the range from 10 to 80 mg/day; or 12 to 70 mg/day; or 14 to 60 mg/day; or 16 to 50 mg/day; or 18 to 40 mg/day; or 19 to 30 mg/day; or 20 mg/day.

In one embodiment, the periodic administration of glatiramer acetate is effected daily.

In another embodiment, the periodic administration of glatiramer acetate is effected twice daily at one half the amount.

In an additional embodiment, the periodic administration of glatiramer acetate is effected once every 3 to 11 days; or once every 5 to 9 days; or once every 7 days; or once every 24 hours.

For each administration schedule of glatiramer acetate, the minocycline may be administered once every 6 to 24 hours; or once every 7 to 22 hours; or once every 8 to 20 hours; or once every 9 to 18 hours; or once every 10 to 16 hours; or once every 11 to 14 hours; or once every 12 hours.

In a further embodiment, the administration of the glatiramer acetate substantially precedes the administration of the minocycline.

In an added embodiment, the administration of the minocycline substantially precedes the administration of the glatiramer acetate.

In one embodiment, the glatiramer acetate and the minocycline may be administered for a period of time of at least 4 days. In a further embodiment, the period of time may be 5 days to 5 years; or 10 days to 3 years; or 2 weeks to 1 year; or 1 month to 6 months; or 3 months to 4 months. In yet another embodiment, the glatiramer acetate and the minocycline may be administered for the lifetime of the subject.

The administration of minocycline or glatiramer acetate may each independently be oral, nasal, pulmonary, parenteral, intravenous, intra-articular, transdermal, intradermal, subcutaneous, topical, intramuscular, rectal, intrathecal, intraocular, buccal or by gavage. For minocycline, the preferred route of administration is oral or by gavage. The preferred route of administration for glatiramer acetate is subcutaneous or oral. One of skill in the art would recognize that doses at the higher end of the range may be required for oral administration.

In one embodiment, the administration of the glatiramer acetate may be subcutaneous, intraperitoneal, intravenous, intramuscular, intraocular or oral and the administration of the minocycline may be oral. In another embodiment, the administration of the glatiramer acetate may be subcutaneous and the administration of the minocycline may be oral.

The subject invention further provides a pharmaceutical composition comprising an amount of glatiramer acetate and an amount of minocycline, wherein the amounts when taken together are effective to alleviate a symptom of a form of multiple sclerosis in a subject.

In one embodiment of the pharmaceutical composition, each of the amount of glatiramer acetate when taken alone and the amount of minocycline when taken alone is effective to alleviate the symptom of multiple sclerosis.

In another embodiment of the pharmaceutical composition, either of the amount of glatiramer acetate when taken alone, or the amount of minocycline when taken alone or each such amount when taken alone is not effective to alleviate the symptom of multiple sclerosis.

In a further embodiment of the pharmaceutical composition, the amount of glatiramer acetate may be in the range from 10 to 600 mg; or 100 to 550 mg; or 150 to 500 mg; or 200 to 450 mg; or 250 to 400 mg; or 300 to 350 mg; or 300 mg.

In an embodiment of the pharmaceutical composition, the amount of glatiramer acetate may be in the range from 10 to 80 mg; or 12 to 70 mg; or 14 to 60 mg; or 16 to 50 mg; or 18 to 40 mg; or 19 to 30 mg; or 20 mg.

Alternatively, the amount of glatiramer acetate in the pharmaceutical composition may be in the range from 50 to 150 mg; or 60 to 140 mg; or 70 to 130 mg; or 80 to 120 mg; or 90 to 110 mg; or 100 mg.

For each amount of glatiramer acetate in the pharmaceutical composition, the amount of minocycline in the pharmaceutical composition may be 50-200 mg; or 60-175 mg; or 70-150 mg; or 80-125 mg; or 90-110 mg; or 95-105 mg; or 100 mg.

The subject invention also provides a package comprising
i) a first pharmaceutical composition comprising an amount of glatiramer acetate and a pharmaceutically acceptable carrier;
ii) a second pharmaceutical composition comprising an amount of minocycline and a pharmaceutically acceptable carrier; and
iii) instructions for use of the first and second pharmaceutical compositions together to alleviate a symptom of a form of multiple sclerosis in a subject.

In an embodiment of the package, the amount of glatiramer acetate may be in the range from 10 to 600 mg; or 100 to 550 mg; or 150 to 500 mg; or 200 to 450 mg; or 250 to 400 mg; or 300 to 350 mg; or 300 mg.

In another embodiment of the package, the amount of glatiramer acetate may be in the range from 10 to 80 mg; or 12 to 70 mg; or 14 to 60 mg; or 16 to 50 mg; or 18 to 40 mg; or 19 to 30 mg; or mg.

Alternatively, the amount of glatiramer acetate in the package may be in the range from 50 to 150 mg; or 60 to 140 mg; or 70 to 130 mg; or 80 to 120 mg; or 90 to 110 mg; or 100 mg.

For each amount of glatiramer acetate in the package, the amount of minocycline in the package may be 50-200 mg; or 60-175 mg; or 70-150 mg; or 80-125 mg; or 90-110 mg; or 95-105 mg; or 100 mg.

The subject invention further provides a pharmaceutical combination comprising separate dosage forms of an amount of glatiramer acetate and an amount of minocycline, which combination is useful to alleviate a symptom of a form of multiple sclerosis in a subject.

In an embodiment of the pharmaceutical combination, each of the amount of glatiramer acetate when taken alone and the amount of minocycline when taken alone is effective to alleviate the symptom of multiple sclerosis.

In an additional embodiment of the pharmaceutical combination, either of the amount of glatiramer acetate when taken alone, the amount of minocycline when taken alone or each such amount when taken alone is not effective to alleviate the symptom of multiple sclerosis.

In a further embodiment, the pharmaceutical combination may be for simultaneous, separate or sequential use to treat the form of multiple sclerosis in the subject.

Formulations of the invention suitable for oral administration may be in the form of capsules, pills, tablets, powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active compound or compounds.

In solid dosage forms of the invention for oral administration (capsules, tablets, pills, dragees, powders, granules and the like), the active ingredient(s) is mixed with one or more pharmaceutically acceptable carriers, such as sodium citrate or dicalcium phosphate, and/or any of the following: fillers or extenders, such as starches, lactose, sucrose, glucose, mannitol, and/or silicic acid; binders, such as, for example, carboxymethylcellulose, alginates, gelatin, polyvinyl pyrrolidone, sucrose and/or acacia; humectants, such as glycerol; disintegrating agents, such as agar-agar, calcium carbonate, calcium phosphate, potato or tapioca starch, alginic acid, certain silicates, and sodium carbonate; solution retarding agents, such as paraffin; absorption accelerators, such as quaternary ammonium compounds; wetting agents, such as, for example, cetyl alcohol and glycerol monostearate; absorbents, such as kaolin and bentonite clay; lubricants, such a talc, calcium stearate, magnesium stearate, solid polyethylene glycols, sodium lauryl sulfate, and mixtures thereof; and coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

Liquid dosage forms for oral administration of the active ingredients include pharmaceutically acceptable emulsions, microemulsions, solutions, suspensions, syrups and elixirs. In addition to the active ingredient(s), the liquid dosage forms may contain inert dilutents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Suspensions, in addition to the active compounds, may contain suspending agents such as ethoxylated isostearyl alcohols, polyoxyethylene sorbitol and sorbitan esters, microcrystalline cellulose, aluminum metahydroxide, bentonite, agar-agar and tragacanth, and mixtures thereof.

The pharmaceutical compositions, particularly those comprising glatiramer acetate, may also include human adjuvants or carriers known to those skilled in the art. Such adjuvants include complete Freund's adjuvant and incomplete Freund's adjuvant. The compositions may also comprise wetting agents, emulsifying and suspending agents, sweetening, flavoring, coloring, perfuming and preservative agents.

Glatiramer acetate may be formulated into pharmaceutical compositions with pharmaceutically acceptable carriers, such as water or saline and may be formulated into eye drops. Glatiramer acetate may also be formulated into delivery systems, such as matrix systems.

This invention will be better understood from the Experimental Details which follow. However, one skilled in the art will readily appreciate that the specific methods and results discussed are merely illustrative of the invention as described more fully in the claims which follow thereafter.

Experimental Details

Experiment 1: Effect of Low Doses of Glatiramer Actetate and Minocycline on Severe Eae

Procedure

In 12-week old C57/BL6 mice, EAE was induced by the use of a low dose of MOG (33-55 peptide) (50 μg) in complete Freund's adjuvant that was not supplemented with mycobacterium. This protocol resulted in mice with severe EAE.

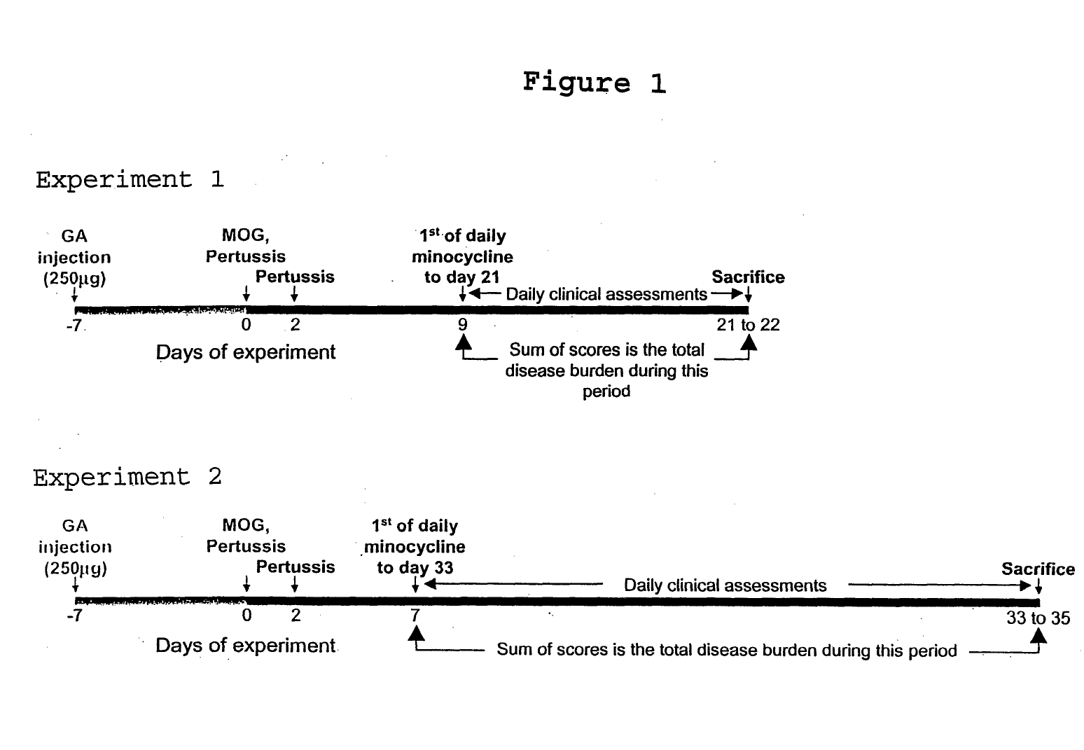

Given that the mechanism of Copaxone® is thought to be one of the generation of Copaxone®-specific Th2 cells, which require time to generate in vivo, Copaxone® was administered to mice as a pre-treatment. Thus, Copaxone® was given as a single injection, 7 days before MOG immunization. Copaxone® was administered in incomplete Freund's adjuvant. A sub-optimal dose of Copaxone® (250 μg) was used. Intraperitoneal pertussis toxin was given on the day of MOG administration and also 2 days after. FIG. 1 presents a layout of the experimental design.

Minocycline was administered intraperitoneally at a sub-optimal dose (25 mg/kg) when the first mouse in the entire experiment developed signs of EAE (See FIG. 1). Minocycline was administered daily until mice were sacrificed.

The following numbers of mice were employed in each group: 10 in the saline vehicle group, and 9 each in the other 3 groups (Copaxone® alone, minocycline alone and combination of Copaxone® and minocycline).

The scale of 0 to 5 that is normally used to assess the degree of disability of EAE is inadequate, since it does not take into account the fact that in many animals only I (rather than 2) of hind or fore limbs are involved, etc. Thus, there could be quite marked differences between 2 mice designated as Grade 2 EAE on the old scale (no forelimb involvement yet): one mouse may have two hind limbs impaired, while the other mouse may have only 1 hind limb involved. Thus, we have developed a new system in order to better differentiate functional outcomes. The scale goes from 1 to 15 and is the sum of the state of the tail and of the 4 limbs.

For the tail:

0—no symptoms

1—half paralysed

2—fully paralysed.

For each of the hind limbs or fore limbs, each assessed separately:

0—no symptoms

1—weak or funny walk

2—dragging but still movable

3—fully paralysed

Thus, a fully paralysed quadriplegic animal would attain a score of 14. A mouse with tail and 3 limbs maximally involved (the fourth being normal) would have a score of 11; in the old scale, this would not have been differentiated easily from a fully paralysed mouse. Death is automatically given a score of 15.

Another parameter that we have employed is the “sum of scores”. This refers to the addition of each daily clinical score over the entire period of the experiment. This is a reflection of the total burden of disease, and takes into account variations in onset of disease, and the daily scores.

Results

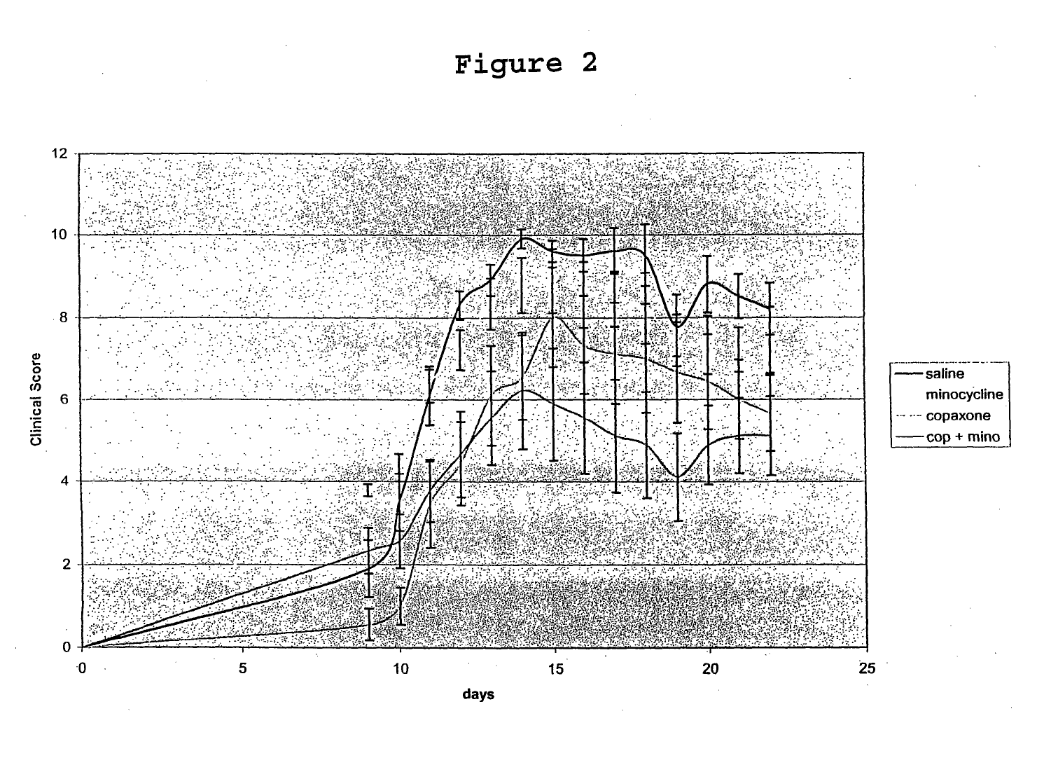

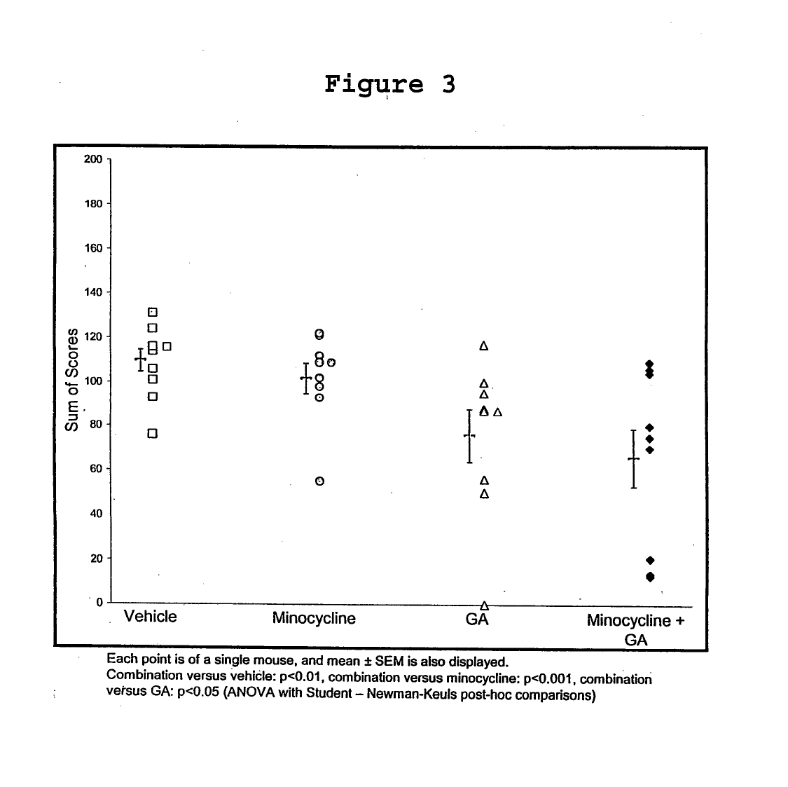

As shown by FIG. 2 and Table 1 below, most animals attained a score of Grade 10 on the new scale, which is indicative of involvement of the forelimbs (that is, at least Grade 4 on the old scale). At best, there might have been a slight benefit when using a sub-optimal dose of Copaxone® alone. Minocycline alone at a sub-optimal dose was ineffective. Interestingly, the combination of Copaxone® plus minocycline resulted in a significant decrease in the clinical scores of EAE (FIG. 2). The sum of scores, representing disease burden throughout the course of the experiment, was plotted from the animals within each group. The results are displayed in FIG. 3 and TABLE 2 below. The combination of minocycline and Copaxone® resulted in a statistically significant reduction of disease burden as compared to vehicle-treated animals.

TABLE 1

| | | Days | | | | | | | | |
|---|---|---|---|---|---|---|---|---|---|---|
| | | 0 | 9 | 10 | 11 | 12 | 13 | 14 | 15 | 16 |
| Scores | 1 | 0 | 0 | 3 | 8 | 8 | 11 | 10 | 10 | 10 |
| | 2 | 0 | 1 | 4 | 8 | 10 | 10 | 10 | 10 | 10 |
| | 3 | 0 | 0 | 3 | 7 | 9 | 10 | 11 | 10 | 10 |
| | 4 | 0 | 0 | 0 | 2 | 6 | 9 | 10 | 10 | 10 |
| | 5 | 0 | 3 | 3 | 5 | 8 | 9 | 10 | 10 | 10 |
| | 6 | 0 | 4 | 5 | 6 | 8 | 8 | 10 | 10 | 9 |
| | 7 | 0 | 6 | 6 | 9 | 8 | 8 | 10 | 10 | 10 |
| | 8 | 0 | 0 | 0 | 4 | 8 | 7 | 10 | 8 | 10 |
| | 9 | 0 | 4 | 5 | 8 | 9 | 9 | 10 | 10 | 10 |
| | 10 | 0 | 1 | 6 | 4 | 9 | 8 | 8 | 8 | 6 |
| | ave | 0 | 2 | 4 | 6 | 8 | 9 | 10 | 10 | 10 |
| | sum | 0 | 0.69 | 0.687 | 0.722 | 0.335 | 0.379 | 0.233 | 0.267 | 0.401 |

1 - Copaxone ®

| | | | | | | | | | |
|---|---|---|---|---|---|---|---|---|---|
| 1 | 0 | 0 | 0 | 0 | 0 | 0 | 3 | 4 | 8 |
| 2 | 0 | 0 | 0 | 4 | 5 | 9 | 8 | 10 | 8 |
| 3 | 0 | 0 | 3 | 7 | 7 | 9 | 10 | 10 | 10 |
| 4 | 0 | 0 | 0 | 0 | 2 | 5 | 8 | 10 | 11 |
| 5 | 0 | 0 | 0 | 0 | 0 | 0 | 0 | 0 | 0 |
| 6 | 0 | 3 | 3 | 4 | 5 | 8 | 8 | 10 | 10 |
| 7 | 0 | 0 | 1 | 8 | 7 | 9 | 8 | 10 | 8 |
| 8 | 0 | 0 | 0 | 2 | 6 | 8 | 6 | 8 | 3 |
| 9 | 0 | 2 | 2 | 6 | 8 | 7 | 8 | 10 | 8 |
| ave | 0 | 1 | 1 | 3 | 4 | 6 | 7 | 8 | 7 |
| sem | 0 | 0.377 | 0.441 | 1.042 | 1.015 | 1.23 | 1.042 | 1.202 | 1.19 |

2 - Minocycline

| | | | | | | | | | |
|---|---|---|---|---|---|---|---|---|---|
| 1 | 0 | 3 | 4 | 8 | 7 | 7 | 8 | 11 | 11 |
| 2 | 0 | 4 | 6 | 5 | 6 | 9 | 10 | 10 | 10 |
| 3 | 0 | 4 | 4 | 6 | 7 | 9 | 10 | 10 | 8 |
| 4 | 0 | 4 | 4 | 7 | 9 | 9 | 10 | 9 | 9 |
| 5 | 0 | 3 | 4 | 7 | 8 | 9 | 10 | 10 | 10 |
| 6 | 0 | 4 | 4 | 4 | 4 | 4 | 4 | 4 | 3 |
| 7 | 0 | 4 | 3 | 6 | 8 | 9 | 8 | 10 | 8 |
| 8 | 0 | 4 | 6 | 7 | 8 | 10 | 10 | 10 | 10 |
| 9 | 0 | 4 | 4 | 7 | 8 | 9 | 9 | 6 | 8 |
| ave | 0 | 4 | 4 | 6 | 7 | 8 | 9 | 9 | 9 |
| sem | 0 | 0.147 | 0.333 | 0.408 | 0.494 | 0.601 | 0.662 | 0.772 | 0.784 |

3 - Combined Therapy with Copaxone and Minocycline

| | | | | | | | | | |
|---|---|---|---|---|---|---|---|---|---|
| 1 | 0 | 2 | 6 | 8 | 8 | 9 | 10 | 10 | 10 |
| 2 | 0 | 1 | 3 | 4 | 6 | 6 | 8 | 8 | 6 |
| 3 | 0 | 4 | 2 | 6 | 4 | 6 | 8 | 8 | 6 |
| 4 | 0 | 4 | 2 | 2 | 2 | 2 | 2 | 0 | 0 |
| 5 | 0 | 0 | 0 | 2 | 2 | 2 | 0 | 0 | 0 |
| 6 | 0 | 3 | 4 | 5 | 4 | 7 | 8 | 6 | 6 |
| 7 | 0 | 3 | 2 | 2 | 6 | 9 | 10 | 10 | 10 |
| 8 | 0 | 0 | 0 | 1 | 0 | 0 | 0 | 2 | 2 |
| 9 | 0 | 4 | 4 | 4 | 10 | 9 | 10 | 9 | 10 |
| ave | 0 | 2 | 3 | 4 | 5 | 6 | 6 | 6 | 6 |
| sum | 0 | 0.553 | 0.648 | 0.76 | 1.054 | 1.144 | 1.432 | 1.379 | 1.365 |

| | | Days | | | | | | Sum of |
|---|---|---|---|---|---|---|---|---|
| | | 17 | 18 | 19 | 20 | 21 | 22 | Scores |
| Scores | 1 | 12 | 12 | 10 | 10 | 10 | 10 | 124 |
| | 2 | 10 | 10 | 7 | 8 | 8 | 8 | 114 |
| | 3 | 10 | 10 | 8 | 10 | 10 | 8 | 116 |
| | 4 | 11 | 11 | 8 | 8 | 8 | 8 | 101 |
| | 5 | 7 | 8 | 8 | 8 | 8 | 9 | 106 |
| | 6 | 10 | 10 | 8 | 10 | 9 | 9 | 116 |
| | 7 | 10 | 10 | 8 | 10 | 10 | 9 | 124 |
| | 8 | 10 | 8 | 8 | 8 | 7 | 5 | 93 |
| | 9 | 10 | 12 | 11 | 12 | 10 | 11 | 131 |
| | 10 | 6 | 4 | 2 | 4 | 5 | 5 | 76 |
| | ave | 10 | 10 | 8 | 9 | 9 | 8 | 110.1 |
| | sum | 0.562 | 0.749 | 0.742 | 0.68 | 0.522 | 0.611 | 5.231 |

1 - Copaxone ®

| | | | | | | | |
|---|---|---|---|---|---|---|---|
| 1 | 8 | 9 | 7 | 5 | 7 | 5 | 56 |
| 2 | 8 | 8 | 8 | 8 | 7 | 5 | 88 |
| 3 | 10 | 10 | 12 | 11 | 9 | 9 | 117 |
| 4 | 10 | 11 | 10 | 10 | 9 | 9 | 95 |
| 5 | 0 | 0 | 0 | 0 | 0 | 0 | 0 |
| 6 | 10 | 10 | 6 | 8 | 7 | 8 | 100 |
| 7 | | 8 | 8 | 8 | 7 | 5 | 87 |
| 8 | 3 | 1 | 2 | 3 | 3 | 5 | 50 |
| 9 | 8 | 6 | 7 | 5 | 5 | 5 | 87 |
| ave | 7 | 7 | 7 | 6 | 6 | 6 | 75.56 |
| sem | 1.227 | 1.323 | 1.236 | 1.168 | 0.972 | 0.928 | 11.7 |

2 - Minocycline

| | | | | | | | |
|---|---|---|---|---|---|---|---|
| 1 | 10 | 11 | 8 | 8 | 7 | 9 | 112 |
| 2 | 10 | 11 | 11 | 11 | 9 | 9 | 121 |
| 3 | 8 | 6 | 6 | 5 | 5 | 5 | 93 |
| 4 | 8 | 10 | 8 | 8 | 7 | 7 | 109 |
| 5 | 10 | 10 | 7 | 7 | 7 | 7 | 109 |
| 6 | 4 | 4 | 4 | 4 | 5 | 3 | 55 |
| 7 | 8 | 6 | 7 | 8 | 8 | 9 | 102 |
| 8 | 10 | 9 | 8 | 9 | 10 | 11 | 122 |
| 9 | 8 | 7 | 8 | 6 | 7 | 7 | 98 |
| ave | 8 | 8 | 7 | 7 | 7 | 7 | 102.3 |
| sem | 0.648 | 0.846 | 0.626 | 0.707 | 0.547 | 0.801 | 6.773 |

3 - Combined Therapy with Copaxone and Minocycline

| | | | | | | | |
|---|---|---|---|---|---|---|---|
| 1 | 10 | 8 | 6 | 8 | 8 | 6 | 109 |
| 2 | 6 | 6 | 6 | 4 | 3 | 3 | 70 |
| 3 | 6 | 6 | 6 | 6 | 6 | 6 | 80 |
| 4 | 0 | 0 | 0 | 0 | 0 | 0 | 14 |
| 5 | 0 | 0 | 0 | 2 | 3 | 2 | 13 |
| 6 | 6 | 6 | 5 | 5 | 5 | 5 | 75 |
| 7 | 10 | 10 | 8 | 8 | 8 | 8 | 104 |
| 8 | 0 | 0 | 0 | 3 | 5 | 8 | 21 |
| 9 | 8 | 8 | 6 | 8 | 8 | 8 | 106 |
| ave | 5 | 5 | 4 | 5 | 5 | 5 | 65.78 |
| sum | 1.379 | 1.296 | 1.06 | 0.964 | 0.92 | 0.964 | 13.29 |

Experiment 2: Effect of Low Doses of Glatiramer Actetate and Minocycline on Moderate EAE
Procedure

The protocol of Experiment 1 was followed. However, the EAE induced was moderate EAE, instead of severe EAE as in Experiment 1. The number of mice in each group was as follows: 8 in the vehicle group, 11 in the minocycline group, 10 in the Copaxone® group, and 9 in the combination group.

Only 1 mouse did not complete the trial and was excluded from analysis. This was saline vehicle mouse #7, which was found dead in the cage on Day 18 of the experiment. This mouse had been bloated for several days and death was presumed to be due to abdominal disturbances of unknown causes.

Results

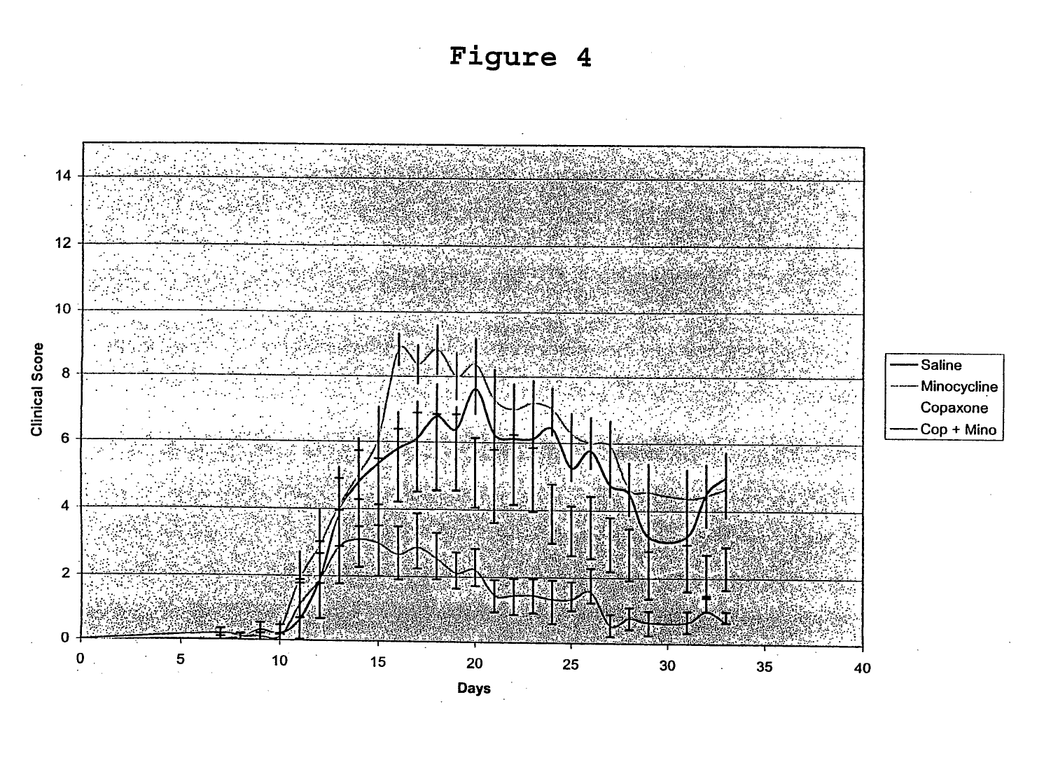

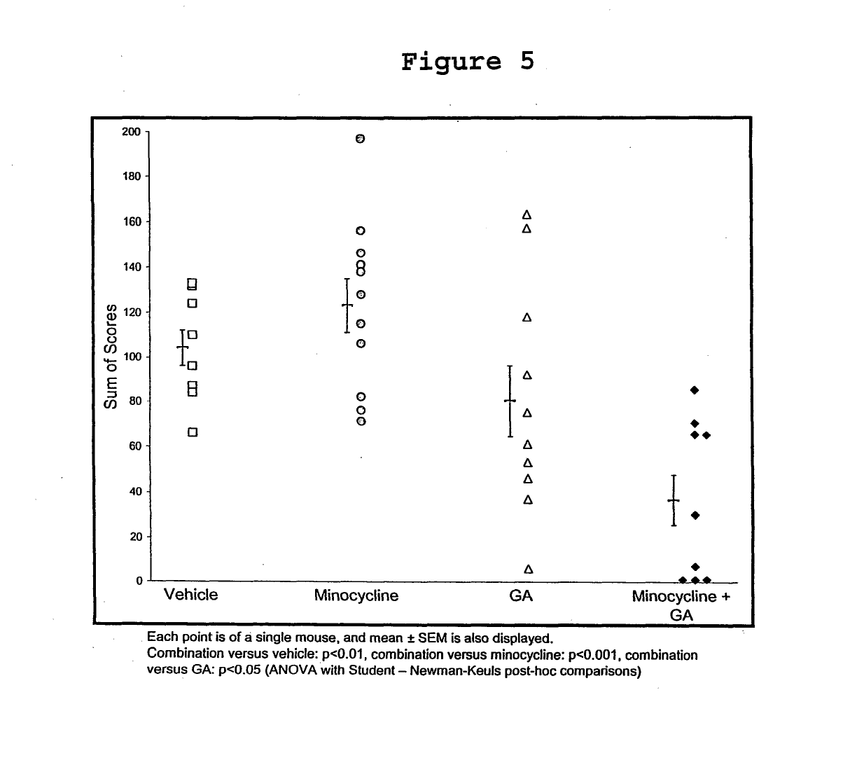

As demonstrated in FIGS. 4 and 5, the maximum disease severity was around Grade 8 (akin to Grade 3 on the old EAE scale). Again, sub-optimal doses of minocycline alone did not elicit a clinical response, while there appears to have been a slight attenuation of disease course in the animals given Copaxone® alone (FIG. 4). As in Experiment 1, the combination of Copaxone® and minocycline resulted in a significant reduction of EAE severity. This was apparent by Day 13, about 6 days after minocycline treatment was initiated. The sum of scores, presented in FIG. 5, showed a significant decrease in disease burden of animals given the combination treatment compared to all other groups.

Experiment 3: Effect of High Doses of Glatiramer Actetate and Minocycline on EAE

Procedure

The procedure of Experiment 1 is followed, except that the dose of Copaxone® is increased to 375 μg, and the dose of minocycline is raised to 37.5 mg/kg. The effects of all treatments versus the control are analyzed histologically (hematoxylin-eosin and Luxol fast blue for evidence of inflammation and demyelination, respectively). Cellular infiltrates are characterized by CD3, CD4, CD8, CD56 and CD19 immunohistochemistry. Blind evaluation (on a qualitative scale from + to ++++) is conducted on the following: the density of perivascular infiltrates, the distance of migration of leukocytes into the CNS parenchyma from blood vessels, and the degree of demyelination. The neuropathological assessments are focused on the optic nerve, lumbar cord and brainstem.

Results

In comparison to Experiments 1-2, higher doses of both drugs alone reduce disease severity in a more apparent manner and restore animals to normal functioning levels. The combination of minocycline and Copaxone® at these doses also significantly reduces disease severity and restores animals to normal functioning levels.

Discussion of EAE Experiments 1-3

In summary, while minocycline and Copaxone® alone at sub-optimal doses did not markedly decrease disease severity, their combination at these doses resulted in a noticeable attenuation of clinical disease. The beneficial effect of the combination treatment was more apparent when the disease severity of animals was moderate (Experiment 2), as compared to severe (Experiment 1). Increasing the doses of minocycline and Copaxone® as in Experiment 3 significantly decreases disease severity and restores animals to normal functioning levels when either drug is administered alone or in combination.

Experiment 4: T Cell Transmigration Across a Fibronectin-Coated Boyden Chamber

Procedure

This experiment is conducted to evaluate whether the combination of minocycline and Copaxone® is more effective at decreasing T cell transmigration across a fibronectin-coated Boyden chamber in vitro than either alone. T lymphocytes of over 90% purity are isolated from the blood of healthy volunteers. For migration assays, 3 μm pore size fibronectin-coated chambers are used. The bottom chamber contains AIM-V medium (serum-free GIBCO® medium) with 1% fetal calf serum (to provide a directional gradient) while 500,000 cells in AIM-V are added to the upper chamber. After 1, 6 or 24 hours, cells in the lower chamber are counted by a Coulter® counter and expressed as percent of the initial cell seeding density; this represents the migratory population. In addition to control conditions (no drug added to the upper chamber), the effect of the following is tested: minocycline alone (50-500 μg/ml, Sigma), Copaxone® alone (1-50 μg/ml, TEVA, Israel) and both in combination. Emphasis is on whether the dose-response of minocycline is shifted (enhanced) by Copaxone®, and whether the previous lack of efficacy for Copaxone® observed in this assay is now reversed by low concentrations of minocycline.

Results

In comparison to Copaxone® alone or minocycline alone, the combination of Copaxone® and minocycline results in comparable or greater attenuation of T cell transmigration.

Experiment 5: Clinical Trial of Relapsing-Remitting Multiple Sclerosis

The purpose of this trial is to compare the treatment of participants with relapsing-remitting multiple sclerosis (RR-MS) with COPAXONE® in combination with minocycline, with treatment with COPAXONE® in combination with placebo. The clinical objective is to evaluate the effect of treatments on MRI variables, clinical evaluations and immunological profile.

The design of this trial is a randomized, double-masked, 2-arm study of COPAXONE® in combination with minocycline versus COPAXONE® in combination with placebo for the treatment of relapsing-remitting multiple sclerosis. Twenty patients with RR-MS who meet the inclusion/exclusion criteria are enrolled per arm. Patients are randomized and receive either 20 mg SQ (subcutaneous) of COPAXONE® daily plus an oral dose of placebo daily or 20 mg SQ of COPAXONE® in combination with 100 mg oral minocycline every 12 hours.

Participant inclusion criteria are as follows: 1) Subjects have clinically definite MS as defined by (Poser C M. et al. New diagnostic criteria for multiple sclerosis: Guidelines for research protocols. Ann. Neurol., 13(3): 227-31, 1983) with disease duration (from onset) of at least 6 months; 2) Subjects have a relapsing-remitting disease course; 3) Subjects have had at least 1 documented relapse within the last year prior to study entry; 4) Subjects have at least 1 and not more than 15 gadolinium (Gd)-enhancing lesions on the screening MRI scan; 5) Subjects must be relapse-free and not have taken corticosteroids (IV, IM and/or PO) within the 30 days prior to the screening visit; 6) Subjects may be male or female. Women of child-bearing potential must use a contraceptive method deemed reliable by the investigator; 7) Subjects are between the ages of 18 and 50 years inclusive; 8) Subjects are ambulatory, with a Kurtzke EDSS score of between 0 and 5 inclusive; and 9) Subjects must be willing and able to give written informed consent prior to entering the study.

Participant exclusion criteria include the following: 1) previous use of injectable glatiramer acetate; 2) previous use of cladribine; 3) previous use of immunosuppressive agents in the last 6 months; 4) use of experimental or investigational drugs, including I.V. immunoglobulin, within 6 months prior to study entry; 5) use of interferon agents or minocycline within 60 days prior to the screening visit; 6) chronic corticosteroid (IV, IM and/or PO) treatment (more than 30 consecutive days) in the 6 months prior to study entry; 7) previous total body irradiation or total lymphoid irradiation (TLI); 8) pregnancy or breast feeding; Patients who experience a relapse between the screening (month −1) and baseline (month 0) visits; 9) significant medical or psychiatric condition that affects the subject's ability to give informed consent, or to complete the study, or any condition which the investigator feels may interfere with participation in the study (e.g. alcohol or drug abuse); 10) a known history of sensitivity to mannitol; 11) contraindication to or known history of sensitivity to tetracyclines; 12) a known history of sensitivity to gadolinium; and 13) inability to successfully undergo MRI scanning.

Subjects appear for a study screening visit, and if they meet all inclusion criteria, they return within 28 days for the baseline visit. Subjects are randomized and receive their first dose of study medication at the baseline visit. Subjects return at months 1, 3, 6, 8 and 9. At the month 9 visit subjects are terminated from the study medication. The treatment duration is 9 months.

The study assessments are performed according to the following Study Task Flow Sheet:

STUDY TASK FLOW SHEET

| | Screen- | Base- | | | | | | Unsch. |
|---|---|---|---|---|---|---|---|---|
| Procedure | ing | line | | | | | | Visit |
| Months* | −1 | 0 | 1 | 3 | 6 | 8 | 9/ET | |
| Informed | X | | | | | | | |
| Consent | | | | | | | | |
| Eligibility | X | X¹ | | | | | | |
| Criteria | | | | | | | | |
| Medical History | X | | | | | | | |
| Historical and | X | X¹ | X | X | X | X | X | X** |
| Concomitant | | | | | | | | |
| medication | | | | | | | | |
| Physical | X | | | | | | X | X** |
| Examination | | | | | | | | |
| Vital Signs | X | X³ | X | X | X | X | X | X |
| ECG | X | | | | | | | X** |
| Chest X-ray*** | X | | | | | | | |
| Neurological | X | X¹ | | | | | X | X** |
| Examination | | | | | | | | |
| Timed 25-foot | X | X¹ | | | | | X | |
| walk | | | | | | | | |
| Evaluation of | X | X¹ | X | X | X | X | X | X** |
| relapse | | | | | | | | |
| Laboratory | X | X¹ | X | X | X | | X | X** |
| Evaluation | | | | | | | | |
| Pregnancy test⁴ | X | X¹ | | | | | | |
| MRI | X⁵ | | X | X | | X | X | |
| Drug Compliance | | X² | X | X | X | | X | X** |
| & Dispensing | | | | | | | | |
| Adverse | | X² | X | X | X | X | X | X** |
| Experiences | | | | | | | | |
| Termination | | | | | | | X | |

¹Performed prior to randomization
²Performed post-randomization
³Pre and post-dose vital signs: (30 minutes post-dose)
⁴For women of childbearing potential. Screening: urine pregnancy test (dipstick) Baseline: serum pregnancy test (β hCG)
⁵Screening MRI must occur 10 days ± 4 days prior to randomization and first-dose.
*A month is defined as 28 days ± 4 days
**Assessments during an unscheduled visit are performed as deemed necessary by the investigator, except vital signs which are performed at each visit and relapse evaluation if the visit is due to subject's complaint of possible relapse.
***The screening chest X-ray may be deferred if a chest X-ray has been performed in the 6 months prior to screening and a report is available. Should the report indicate abnormal findings, the chest x-ray must be repeated.

Efficacy Assessment
MRI Evaluation

The subjects undergo MRI of the brain at screening and at months 1, 3, 8 and 9 or early termination (if the patient has been in the trial for at least 3 months). The following is measured:
Total number of new T1-Gd enhancing lesions
Total number of new T2 lesions
Total number of active (new or enlarging) T2 lesions
Total volume of T2 lesions (screening & termination only)
Number of new lisions identified at month 1 and month 3 which become persistent/chronic T1 hypointensities at month 9

In the event that a subject receives steroid treatment for a relapse, as allowed by the protocol, a scheduled MRI is performed and not delayed.

All MRI data is interpreted in a blinded fashion by the MRI Analysis Center (MRI-AC). MRI is performed ±4 days prior to/after each visit requiring MRI (months 1, 3, 8 and study completion at month 9).

Both the Treating and Examining neurologists remain blinded to the results of all MRI scans. The only exception is that the screening MRI scan may be reviewed by the Treating neurologist to determine if a patient meets inclusion criteria. However, patients are not randomized into the trial until the MRI Analysis Center confirms the screening MRI is received in acceptable condition and the patient meets MRI inclusion criteria.

Efficacy Endpoint

The primary end-point for this study is the total number of enhancing lesions measured at months 8 and 9. The analysis of this end-point utilizes the Quasi-Likelihood (over-dispersed) Poisson Regression (SAS® PROC GENMOD employing DIST=POI and DSCALE in the options section of the MODEL statement) with an “offset” based on the log of exposure. Screening lesion count is used as a covariate. Treatment and center effects are included in the model. The center-by-treatment interaction term is tested using the −2 log likelihood ratio test. The treatment-by-center interaction is included in the principal analysis model if it is found to be statistically significant (i.e. if p<0.10).

The Hierarchy Approach is used to control for multiplicity, resulting from the planned secondary end-points significance testing. The secondary endpoints, given in the hierarchical order of the statistical analysis are outlined below. Secondary outcome assessments compare the two study groups in MRI parameters as measured at months 8 and 9 visits: 1) the total number of new T1 Gd-enhancing lesions; 2) the total number of new T2 lesions; and 3) the change from baseline to termination in total volume of T2 lesions. The number of new lesions of MRI scans counted in month 9, with reference to the previous scan, is analyzed similarly to the primary endpoint. The change from baseline to termination in total volume of T2 lesions is analyzed applying the Analysis of Covariance (ANCOVA, SAS. GLM procedure) comparing the adjusted means of the changes observed in the two treatment groups. The model includes the following fixed effects: treatment group, center and baseline volume measurement. The treatment-by-center interaction is included in the model if it is found to be statistically significant (i.e. if p<0.10). Rank transformation is implemented to the volume changes in case of significant (i.e. if p<0.001—Shapiro-Wilk's test) deviation from normality.

Exploratory outcome assessments compare the two study groups in the following parameters: 1) the number of new lesions (Gd-enhancing and/or new PD/T2 lesions) identified at month 1 (compared to screening) and month 3 (compared to month 1) which become persistent/chronic T1 hypointensities (black holes) at month 9; 2) Treatment effect evolution as measured by MRI metrics at baseline and at months 1, 3, 8 and 9; 3) Changes from baseline to termination in EDSS; 4) The total number of confirmed relapses; and 5) Change from baseline to termination in Timed 25-Foot Walk test. MRI count data are analyzed using the over-dispersed Poisson regression as outlined for the primary end-point. MRI volume changes from baseline to termination are analyzed using an analysis of covariance as described for the changes in the T2 lesions volume. Repeated measures analysis designed to elucidate the time course of the drug effect is also performed for all MRI parameters. The changes from baseline to termination in the EDSS and in the Timed 25-Foot Walk test are analyzed using an analysis of covariance as outlined for the change from baseline to termination in total volume of T2 lesions.

Neurological Evaluations

Each patient is evaluated by an Examining Neurologist for the neurological exam and relapse evaluation. The Treating Neurologist sees the subject for relapse confirmation, based on the Examining Neurologists, findings, and may prescribe steroid treatment if warranted. The Examining Neurologist performs all neurological evaluations throughout the study. FS, EDSS score, and Timed 25-Foot Walk is assessed based on standardized neurological examination and slightly, modified FS and EDSS (Neurostatus L. Kappos, Dept. of Neurology, University Hospital, CH-4031/Base1),

Relapse Evaluation

Relapse Definition (Attack)

A relapse is defined as the appearance of one or more new neurological abnormalities or the reappearance of one or more previously observed neurological abnormalities.

This change in clinical state must last at least 48 hours and be immediately preceded by a relatively stable or improving neurological state of at least 30 days. This criterion is different from the clinical definition of exacerbation “at least 24 hours duration of symptoms” (Poser C M. et al. New diagnostic criteria for multiple sclerosis: Guidelines for research protocols. Ann. Neurol., 13(3): 227-31, 1983). Since “in study” exacerbation definition must be supported by an objective neurological evaluation (see next paragraph), a neurological deficit must sustain long enough to eliminate pseudo exacerbations.

An event is counted as a relapse only when the subject's symptoms are accompanied by observed objective neurological changes, consistent with:
a) an increase of at least 0.5 in the EDSS score or one grade in the score of two or more of the seven FS; or,
b) two grades in the score of one of FS as compared to the previous evaluation.

The subject must not be undergoing any acute metabolic changes such as fever or other medical abnormality. A change in bowel/bladder function or in cognitive function must not be entirely responsible for the changes in EDSS or FS scores.

Subject Evaluation by the Examining Neurologist

A complete neurological assessment is performed at screening, baseline, and month 9 (or early termination visit). A complete neurological assessment is also performed during a schedule or unscheduled visit when the subject's complaint is suggestive of a relapse.

Relapse Determination by the Treating Neurologist

The decision as to whether the neurological change is considered a confirmed relapse is made by the Treating Physician, based on EDSS/FS scores assessed by the Examining Physician.

Follow-up visits to monitor the course of the relapse are made at the Treating Physician's discretion, in addition to the assessment at the next scheduled visit, but the neurological assessments are performed by the Examining Physician.

Relapse Evaluation Procedures

Subjects is instructed to telephone their study site immediately should any symptoms suggestive of a relapse appear.
The subject is examined within 7 days after a symptomatic period of ≧48 hs:
   The Treating Neurologist evaluates the subject once any symptom suggestive of a relapse occurs.
   In the presence of symptoms suggestive of a relapse during a scheduled or unscheduled visit, the Treating Neurologist will refer the patient to the Examining Neurologist.
      Relapse Treatment

The investigator makes the decision whether or not corticosteroids should be administered for the treatment of the relapse. A fixed dose of 1 g/day of I.V. methylprednisolone (Solumedrol®) for 3 consecutive days maximum is the treatment allowed in this protocol. No tapering off is allowed.

Study Medication

Subjects are treated with a daily dose of 26 mg of GA in combination with a twice-daily dose of 100 mg of minocycline or with a daily dose of 20 mg of GA in combination with a placebo. GA is supplied by Teva Pharmaceitical Industries Ltd, Israel. The minocycline and placebo are supplied by Novopharm Ltd, Canada.

Results

Patients treated with the COPAXONE® and minocycline combination exhibit a comparable or greater reduction in T1 and T2 Gd-enhancing lesions and other lesions, as compared to the group receiving COPAXONE® and placebo. Additionally, the group receiving the COPAXONE® and minocycline combination demonstrate a comparable or greater reduction in the number of relapses per year as compared with the group receiving COPAXONE® and placebo.

1. A method of treating a subject afflicted with a form of multiple sclerosis comprising periodically administering to the subject an amount of glatiramer acetate and an amount of minocycline, wherein the amounts when taken together are effective to alleviate a symptom of the form of multiple sclerosis in the subject so as to thereby treat the subject.

2. The method of claim 1, wherein the form of multiple sclerosis is relapsing-remitting multiple sclerosis.

3. The method of claim 1, wherein the subject is a human being.

4. The method of claim 1, wherein each of the amount of glatiramer acetate when taken alone, and the amount of minocycline when taken alone is effective to alleviate the symptom of the form of multiple sclerosis.

5. The method of claim 1, wherein either the amount of glatiramer acetate when taken alone, the amount of minocycline when taken alone or each such amount when taken alone is not effective to alleviate the symptom of the form of multiple sclerosis.

6. The method of claim 1, wherein the symptom is the frequency of relapses, the frequency of clinical exacerbation, or the accumulation of physical disability.

7. The method of claim 1, wherein the amount of glatiramer acetate is in the range from 10 to 600 mg/week.

8. The method of claim 7, wherein the amount of glatiramer acetate is 300 mg/week.

9. The method of claim 1, wherein the amount of glatiramer acetate is in the range from 50 to 150 mg/day.

10. The method of claim 9, wherein the amount of glatiramer acetate is 100 mg/day.

11. The method of claim 1, wherein the amount of glatiramer acetate is in the range from 10 to 80 mg/day.

12. The method of claim 11, wherein the amount of glatiramer acetate is 20 mg/day.

13. The method of claim 1, wherein the amount of minocycline is 100 to 400 mg/day.

14. The method of claim 13, wherein the amount of minocycline is 200 mg/day.

15. The method of claim 1, wherein the periodic administration of glatiramer acetate is effected daily.

16. The method of claim 1, wherein the periodic administration of glatiramer acetate is effected twice daily at one half the amount.

17. The method of claim 1, wherein the periodic administration of glatiramer acetate is effected once every 5 to 9 days.

18. The method of claim 1, wherein the periodic administration of minocycline is effected once every 6 to 24 hours.

19. The method of claim 18, wherein the periodic administration of minocycline is effected once every 12 hours.

20. The method of claim 1, wherein the administration of the glatiramer acetate substantially precedes the administration of the minocycline.

21. The method of claim 1, wherein the administration of the minocycline substantially, precedes the administration of the glatiramer acetate.

22. The method of claim 1, wherein the administration of the glatiramer acetate is effected subcutaneously, intraperitonealiy, intravenously, intramuscularly, intraocularly or orally and the administration of the minocycline is effected orally.

23. The method of claim 22, wherein the administration of the glatiramer acetate is effected subcutaneously and the administration of the minocycline is effected orally.

24. A pharmaceutical composition comprising an amount of glatiramer acetate and an amount of minocycline, wherein the amounts when taken together are effective to alleviate a symptom of a form of multiple sclerosis in a subject.

25. The pharmaceutical composition of claim 24, wherein each of the amount of glatiramer acetate when taken alone and the amount of minocycline when taken alone is effective to alleviate the symptom of multiple sclerosis.

26. The pharmaceutical composition of claim 24, wherein either of the amount of glatiramer acetate when taken alone, or the amount of minocycline when taken alone or each such amount when taken alone is not effective to alleviate the symptom of multiple sclerosis.

27. A package comprising
a) a first pharmaceutical composition comprising an amount of glatiramer acetate and a pharmaceutically acceptable carrier;
b) a second pharmaceutical composition comprising an amount of minocycline and a pharmaceutically acceptable carrier; and
c) instructions for use of the first and second pharmaceutical compositions together to alleviate a symptom of a form of multiple sclerosis in a subject.

28-32. (canceled)
